(19) United States
(12) Patent Application Publication
(10) Pub. No.: US 2024/0197501 A1
(43) Pub. Date: Jun. 20, 2024

(54) PATIENT-SPECIFIC HYBRID STENT

(71) Applicant: VisionAir Solutions, LLC, Cleveland, OH (US)

(72) Inventors: Keith Grafmeyer, Cleveland, OH (US); William Flickinger, Cleveland, OH (US)

(21) Appl. No.: 18/389,644

(22) Filed: Dec. 19, 2023

Related U.S. Application Data
(60) Provisional application No. 63/433,613, filed on Dec. 19, 2022.

(51) Int. Cl. A61F 2/90

(52) U.S. Cl. CPC A61F 2/90; A61F 2002/043

(57) ABSTRACT

A method for configuring a stent provides a patient anatomical lumen model and provides a target anatomical lumen model. The method determines a stent strength profile for the stent based on the patient anatomical lumen model and the target anatomical lumen model. The method determines a stent model including an elastomeric body model and a scaffold model based on the stent strength profile. The method then transmits instructions effective to construct the stent using the stent model. The stent has an elastomeric body corresponding to the elastomeric body model and a scaffold, having metallic material, corresponding to the scaffold model.

### PRIORITY

This patent application claims priority from provisional U.S. patent application No. 63/433,613, filed Dec. 19, 2022, entitled, “PATIENT-SPECIFIC HYBRID AIRWAY STENT,” and naming Keith Grafmeyer et al. as inventors, the disclosure of which is incorporated herein, in its entirety, by reference.

### FIELD

Illustrative embodiments of the invention generally relate to stents and, more particularly, various embodiments of the invention relate to customized stents for a patient.

### BACKGROUND

When an inner cavity of an anatomical tubular structure, also known as an anatomical lumen, is at risk for narrowing or closing, a medical professional may insert a stent. FIG. 1 shows a human airway having an anatomical lumen including a trachea and a branching bronchi structure. The airway includes cross-sections which vary in diameter and segments which vary in length and curvature. While airways may have a similar topology, airways differ in size and shape from person to person. An improperly sized or shaped stent may be ineffective for opening the airway, or may cause additional problems. For example, a straight stent placed in a curved airway may irritate the airway by applying unintended pressure.

### SUMMARY OF VARIOUS EMBODIMENTS

In accordance with one embodiment, a method for constructing a stent provides a patient anatomical lumen model and provides a target anatomical lumen model. The method determines a stent strength profile for the stent based on the patient anatomical lumen model and the target anatomical lumen model. The model determines a stent model including an elastomeric body model and a scaffold model based on the stent strength profile. The model transmits instructions to an electronic control system effective to construct the stent using the stent model, the stent including an elastomeric body corresponding to the elastomeric body model and a scaffold corresponding to the scaffold model, the scaffold comprising a metallic material.

The stent model may have a plurality of cross-sections and the stent strength profile may have a strength parameter with varying values corresponding to each of the plurality of cross-sections. The strength parameter may be a radial stiffness parameter or a radial force parameter.

Determining the stent model may include determining an elastomeric body model length based on the stent strength profile and determining a scaffold length based on the stent strength profile.

Determining the elastomeric body model and the scaffold model based on the stent strength profile may include determining a position of the elastomeric body model relative to the scaffold model.

In some embodiments, the method constructs the stent in response to transmitting the instructions.

Determining the stent strength profile may include determining an anatomical abnormality position and determining the elastomeric body model and the scaffold model based on the stent strength profile may include determining a scaffold model position relative to the elastomeric body model based on the anatomical abnormality position.

In accordance with another embodiment, a patient-specific stent has an elastomeric body and a scaffold coupled to the elastomeric body and including a metallic material. The patient-specific stent is configured to include a stent strength profile along a length of the patient-specific stent, the stent strength profile including an elevated strength section.

The patient-specific stent may have a first cross-section formed of the elastomeric body and a second cross-section parallel to the first cross-section, and a radial stiffness of the second cross-section is greater than a radial stiffness of the first cross-section.

The stent strength profile may be a function of an elastomeric body length relative to a scaffold length.

A location of the elevated strength section of the stent strength profile may be a function of a scaffold position relative to the elastomeric body.

The elastomeric body may comprise a least one of silicone or polyurethane.

The stent strength profile may include a first radial stiffness parameter corresponding to an anatomical abnormality cross-section of the stent and a second radial stiffness parameter corresponding to another cross-section of the stent. The first radial stiffness parameter is at least two times greater than the second radial stiffness parameter.

The stent strength profile may have a varying radial stiffness as a function of at least one of: a varying thickness of the elastomeric body, a difference between an elastomeric body length and a scaffold length, a varying composition of the elastomeric body or the scaffold, a varying geometric pattern of the scaffold, or a position of the scaffold relative to the elastomeric body.

In accordance with another embodiment, a method for treating an anatomical lumen determines a first stent strength profile based on a patient anatomical lumen and a target anatomical lumen, the first stent strength profile including a first elevated strength section. The method constructs a first stent based on the first stent strength profile, the first stent including an elastomeric body coupled to a metallic scaffold. The method implants the first stent in the patient anatomical lumen, wherein the elevated strength section of the stent strength profile is configured to align with an anatomical anomaly. The method removes the stent. The method determines a second stent strength profile based on the first stent strength profile and the patient anatomical lumen, the second stent strength profile including a second elevated strength section. The method then constructs a second stent based on the second stent strength profile.

Illustrative embodiments of the invention are implemented as a computer program product having a computer usable medium with computer readable program code thereon. The computer readable code may be read and utilized by a computer system in accordance with conventional processes. In accordance with another embodiment, a computer program product for configuring a stent including program code for providing a patient anatomical lumen model; program code for providing a target anatomical lumen model; program code for determining a stent strength profile for the stent based on the patient anatomical lumen model and the target anatomical lumen model; program code for determining a stent model including an elastomeric body model and a scaffold model based on the stent strength profile; and program code for transmitting instructions effective to construct the stent using the stent model, the stent including an elastomeric body corresponding to the elastomeric body model and a scaffold corresponding to the scaffold model, the scaffold comprising a metallic material.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 shows a typical human airway.

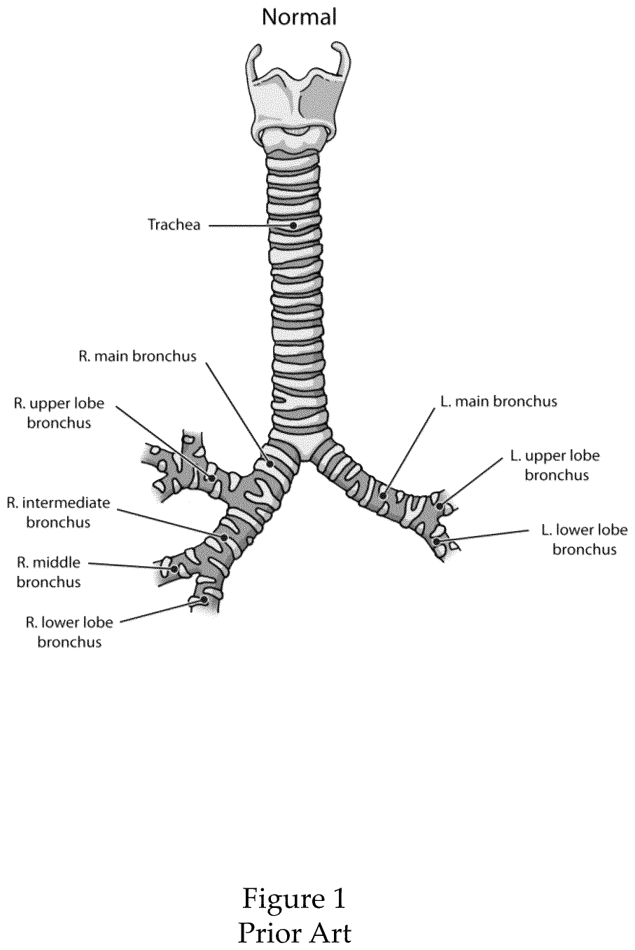

FIG. 2 schematically shows a side view of a stent in an anatomical lumen in accordance with various embodiments.

FIG. 3 schematically shows a partial stent cross-section in accordance with various embodiments.

FIG. 4 schematically shows a stent with multiple scaffolds in accordance with various embodiments.

FIG. 5 schematically shows the stent inserted in an anatomical lumen adjacent to an anatomical abnormality in accordance with various embodiments.

FIG. 6 schematically shows a stent with an elastomeric body having three openings in accordance with various embodiments.

FIG. 7 is a graph showing strength parameters of a stent strength profile in accordance with various embodiments.

FIG. 8 is a flowchart showing a process for configuring the stent in accordance with various embodiments.

FIG. 9 is a flowchart showing a process for constructing a stent in accordance with various embodiments.

FIG. 10 is a flowchart showing a process for inserting a series of stents in accordance with various embodiments.

FIG. 11 is a box diagram schematically showing a computing device in accordance with various embodiments.

### DESCRIPTION OF ILLUSTRATIVE EMBODIMENTS

In illustrative embodiments, a stent is comprised of multiple materials and has a strength profile such that the stent may resist or correct lumen narrowing or obstruction. The stent may be shaped in such a way that substantially conforms to the contour or desired contour of the anatomical lumen into which the stent will be placed. Details of illustrative embodiments are discussed below.

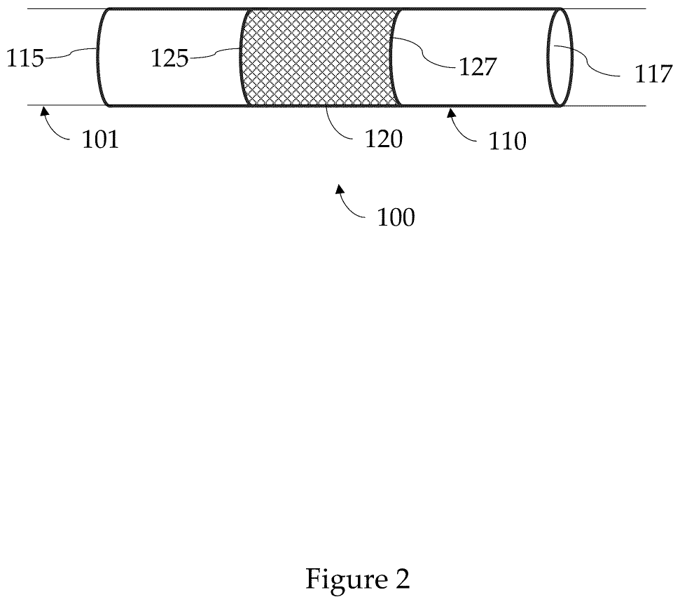

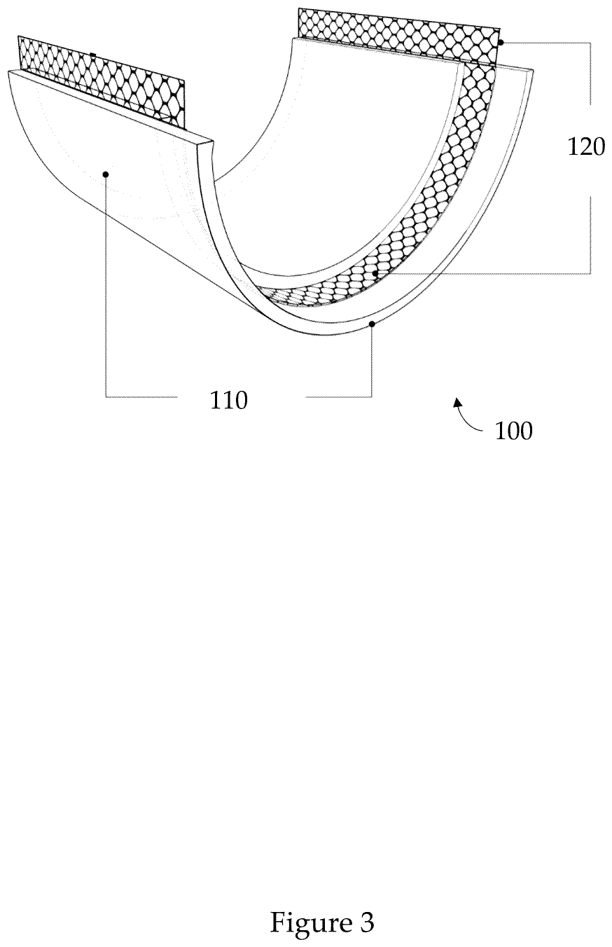

FIGS. 2 and 3 schematically show a stent 100 positioned within an anatomical lumen 101 and configured to expand or resist narrowing of the anatomical lumen 101 in accordance with various embodiments. Embodiments below illustrate stent design for a human airway. Other embodiments may include stents designed for animals, or stents designed for other anatomical lumens, such as vascular stents, colonic stents, biliary stents, ureteral stents, or esophageal stents, among other things.

It should be appreciated that, for both healthy and unhealthy people, anatomical lumens may not be perfectly cylindrical and cross-sections thereof may not be perfectly circular. As illustrated, the anatomical lumen 101 forms a tube-like structure with generally circular cross-sections, but other anatomical lumens may have different characteristics. For example, other anatomical lumens have oval cross-sections, U-shaped cross-sections, or branching airways, among other things. In some cases, the anatomical lumen 101 may have a partial obstruction or narrowing due to an abnormality, such as a tumor, among other things.

The stent 100 includes multiple elongated bodies including an elastomeric body 110 and a scaffold 120. In the illustrated embodiment, the multiple bodies of the stent 100 are shaped like elongated tubes with cross-sections, axial curvature, diameter substantially corresponding to a patient's anatomical lumen 101. Each of the elastomeric body 110 and the scaffold 120 may extend from a first end to a second end and comprise a central opening extending from the first end through to the second end. In other embodiments, the stent 100 may also be shaped to include more than two openings, such as for insertion into two branching portions of an anatomical lumen.

The stent 100 includes an elastomeric body 110 having openings at ends 115 and 117. The outer surface of the elastomeric body 110 may be based on the shape and size of the anatomical lumen 101, as illustrated. For example, the diameter of the stent 100 may correspond to the varying diameters of the anatomical lumen 101. In another example, some or all of the cross-sections of the stent 100 may be configured to expand the anatomical lumen 101 some percentage to correct a narrowing or closure of the anatomical lumen 101. By inserting the stent 100 into position within the anatomical lumen 101, the stent 100 expands the anatomical lumen 101. The shape of the stent 100 along the longitudinal direction of the stent 100 (e.g., straightness, curvature, degree of curvature) may be chosen relative to the anatomical structure, and may be between an actual anatomy and a desired anatomy, as discussed elsewhere herein

The elastomeric body 110 may be comprised of an elastomer, such as, silicone or polyurethane, among other things. The elastomeric body 110 has a thickness which may be uniform or varying throughout either a cross-section of the elastomeric body 110, or different segments of the elastomeric body 110. The variation of the thickness of the elastomeric body 110 may be as a function of position along a generally lengthwise direction of the stent 100. The thickness may vary depending on the desired radial strength or radial stiffness in various places. Among other things, the thickness of the elastomeric body 110 may have a range inclusive of 0.25 mm to 2.0 mm.

The stent 100 further includes a scaffold 120 having openings at ends 125 and 127. As illustrated, the scaffold 120 may be located in a middle section of the stent 100. In other embodiments, the scaffold 120 may be located toward an end of the elastomeric body 110, among other things. The scaffold 120 may reside within or be embedded inside the elastomeric body 110. In some embodiments, a portion of the scaffold 120 is positioned within the elastomeric body 110. In some embodiments, an outer surface of the scaffold 120 protrudes from the outer surface of the elastomeric body 110. The stent 100 may include sections with only the scaffold 120 and not the elastomeric body 110.

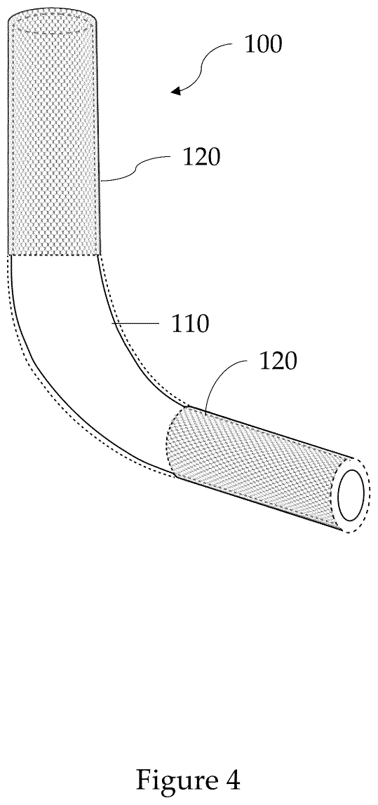

In some embodiments, the stent 100 includes more than one scaffold 120 positioned in different segments of the elastomeric body 110, as illustrated in FIG. 4. In some embodiments, the stent 100 may have multiple scaffolds 120 each having different properties. The provision of different scaffold regions may be provided as a function of scaffold design parameters such as the dimensions of struts, number of struts, or connection pattern among struts, among other things. In some embodiments, the scaffold 120 exerts a reinforcing force on the elastomeric body 110, adding rigidity to the elastomeric body 110.

The scaffold 120 may include an outer surface cross-section with a diameter based on the shape of the anatomical lumen 101. The scaffold 120 may be constructed with the same axial curvature as the anatomical lumen 101. In this way, the scaffold 120 provides the largest possible opening without exerting an unnecessary bending force on the anatomical lumen 101, and the lumen 101 does not exert bending stress on the scaffold 120, avoiding fatiguing the scaffold 120.

In some embodiments, the scaffold 120 is comprised of a metallic structure, such as a metal mesh, among other things. The metal may include stainless steel, platinum-chromium, or cobalt-chromium, nitinol (which is a nickel-titanium alloy), cobalt chrome molybdenum alloy, titanium, or alloys of titanium, among other metallic materials known to those of skill in the art of stent design. The material such as nitinol may have super elastic properties or shape memory properties or both

In some embodiments, the scaffold 120 may be collapsible or able to be temporarily reduced in dimension, especially in its radial or cross-sectional dimensions. The scaffold 120 may be collapsed or temporarily reduced in size while the stent 100 is being inserted into the anatomical lumen 101, and then allowed to expand or caused to expand when the stent 100 is properly positioned.

In some embodiments, it is possible that a stent 100 may be manufactured to have a natural undeformed shape that, following along the longitudinal direction of the stent 100, is generally curved, perhaps corresponding to anatomical features or goals as described elsewhere herein. Nevertheless, that same stent 100, for purposes of implantation, may be deformed to a shape that is generally straight, or may be closer to straight than the natural shape of the stent 100. Such deformation may be within the elastic (recoverable) range of deformation of the stent 100.

The scaffold 120 may be collapsible into a sheath for deployment over a catheter significantly smaller than the anatomical lumen 101 and expanded stent diameter. The scaffold 120 may be collapsed while the stent 100 is being inserted into the anatomical lumen 101, and then expanded when the stent 100 is properly positioned. When placed inside a patient's body, the scaffold 120 may have a thickness which may be uniform or varying throughout either a cross-section of the scaffold 120, or different segments of the scaffold 120. Among other things, the thickness of the scaffold 120 may have a range inclusive of 25% to 120% of the thickness of the molded body.

The length of the elastomeric body 110 may be different than the length of the scaffold 120. In the illustrated embodiment, the length of the scaffold 120 is equal to the length of the elastomeric body 110. In other embodiments, the lengths of the bodies may differ. For example, the length may be less than 80% of the length, 90% of the length, or 95% of the length, among other things.

In some embodiments of the invention, the scaffold may comprise filaments, such that the filaments are longer (with respect to their diameter) than is typically the case for struts. In fact, the filaments, as made by 3DP using a powder bed, may be unjoined to other filaments. The filaments may even pass over and under each other in a woven configuration. As another alternative, the filaments may have a substantial length as discrete filaments but may also have a region in which they have joints to other filaments or struts. It can be noted because it is likely that the filaments would eventually be encased in an elastomeric body 110, the structural strength of the filaments might be less important than would be the case for a free-standing structure.

The thickness of the elastomeric body 110 also may affect the ease or ability of the stent to collapse or be squeezed such as for being stored inside an endoscopic instrument for purposes of being introduced into a patient's body and to be restored to approximately its original dimensions when released.

Reference to thickness may refer to local thickness of the stent or of the elastomeric body 110. Such thickness does not have to be identical everywhere in the stent. The local thickness may be either uniform or nonuniform among various places in the stent. If the thickness of the elastomeric body 110, or of the scaffold 120, varies along the lengthwise direction of the stent, that may provide different radial strength in different places on the stent. This thickness can be correlated with expected properties of the tissue adjacent to the stent, which in individual patients could include the presence of tumors or other anatomical abnormalities.

One possibility is that the thickness of the stent 100 or of the elastomeric body 110 may be circumferentially uniform at a local circumference at a cross-section taken perpendicular to the local longitudinal direction of the stent, but this thickness may be different from one location along the length of the stent 100 to another location along the length of the stent 100. For example, it is possible that a stent 100 may be generally tapered having one end wider and an opposed end narrower. It may be desirable that the thickness of the elastomeric body 110 or the thickness of the stent 100 in general be greater in the portion of the stent 100 that is wider and may be lesser in the portion of the stent 100 that is narrower. If the stent is formed by a molding process, the local thickness of the stent 100 may be determined by the distance between corresponding parts of the mold during the molding process, such as distance between the shell of the mold and the insert or core that occupies (temporarily, during the molding process) the lumen space of the mold and of the stent.

The stent 100 is configured to have varying strength parameters over the length of the stent 100, also known as a stent strength profile. The stent strength profile may refer to the strength at cross-sections of the stent 100 as a function of position along the longitudinal axis of the stent 100, where different cross-sections are configured to exhibit different strengths based on features of the anatomical lumen 101.

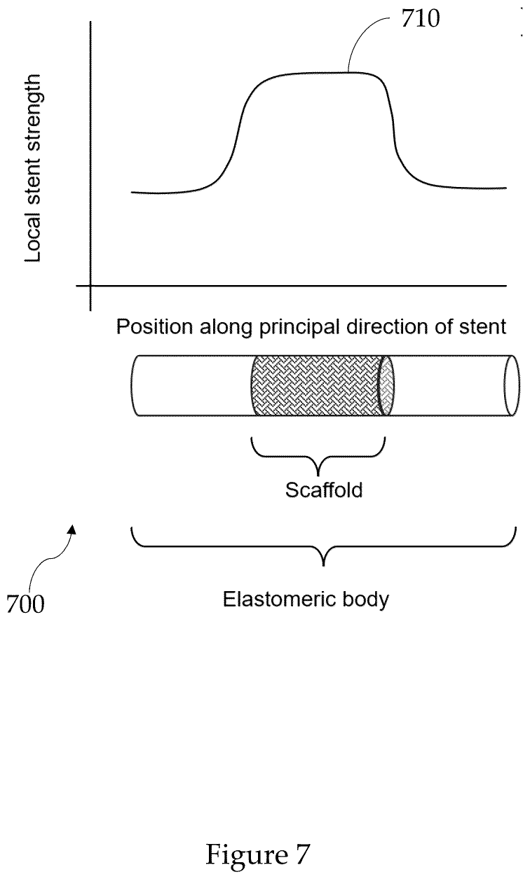

FIG. 7 shows a graph 700 illustrating a stent strength parameter of the stent strength profile at cross-sections over the length of the illustrated stent 100. In the illustrated embodiment, the stent 100 has an elevated strength section where the stent 100 has the scaffold 120 in addition to the elastomeric body 110. The elevated strength section may correspond to a section of the anatomical lumen 101 where an anatomical anomaly is located. In some embodiments, the elevated strength section may correspond to strength parameter (i.e., radial stiffness, radial strength, radial force) at least two times greater than the same strength parameter corresponding to a non-elevated strength section of the stent 100. In some embodiments, the strength parameter of the elected strength section may be 1.5 times or three times greater than same strength parameter corresponding to a non-elevated strength section of the stent 100.

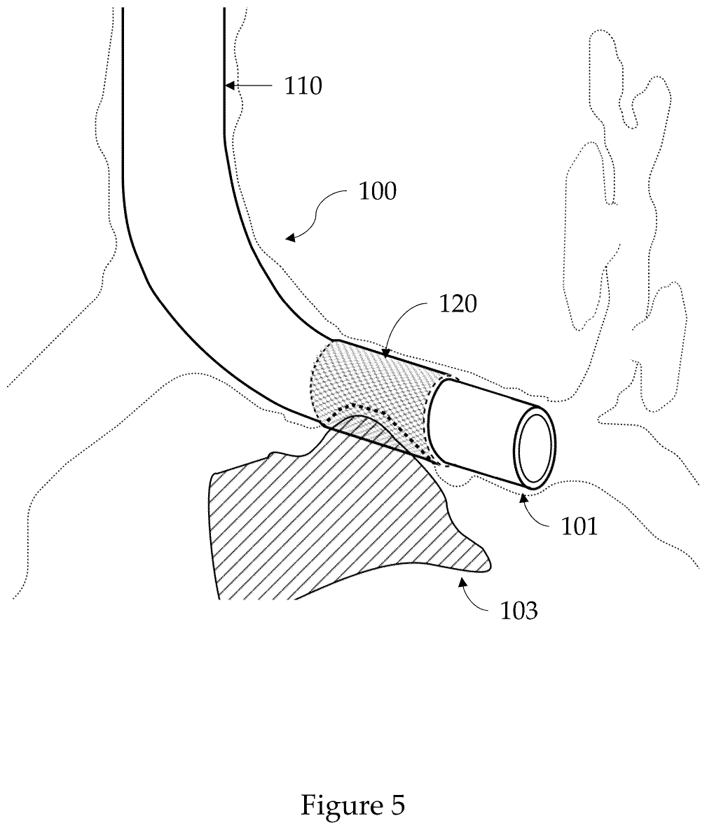

The parameters of the stent strength profile may include radial stiffness, which may be the change in stent diameter as a function of uniformly applied external radial pressure. In some embodiments, radial stiffness may be calculated as the external radial pressured divided by the change in stent diameter. The radial stiffness of the stent 100 may correspond to the ability of the stent 100 to resist the compressive force of the anatomical lumen 101, or other anatomical structures, such as a tumor, on the stent 100, as shown in FIG. 5. In general, a network made of metallic wire is likely to contribute more to the radial stiffness of a stent than is contributed by a layer of elastomeric material such as silicone, because silicone is a fairly soft material. Combining the metallic network and the elastomeric material would provide larger radial stiffness or strength than either one alone.

The parameters of the stent strength profile may also include radial strength, which may be the pressure at which a stent experiences irrecoverable deformation. The radial strength may be related to the stability of the stent against buckling. The stability against buckling may in turn be related to the elastic properties of the material(s) of which the stent is made, and to its local thickness and other geometric and dimensional properties. This may be relevant if a localized applied force is nonuniform such as may derive from the presence of a specific tumor in a specific patient. For example, a section of the stent 100 with a metallic mesh scaffold 120 is likely to have greater radial strength than a section of a stent made of a polymeric material, and the combination of the elastomeric body 110 and scaffold 120 is likely to have greater radial strength than either one alone.

The parameters of the stent strength profile may include radial force, which may be the output of radial loading that equals the radial pressure times the stent cylindrical area, as defined in Section 3.1.6 of ASTM F3067-14 Standard Guide for Radial Loading of Balloon-Expandable and Self-Expanding Vascular Stents (Reapproved 2021) (hereinafter “ASTM Standard”).

The strength parameters of the stent strength profile of the stent 100 may be measured as described in the ASTM Standard, among other methods. For example, the stent 100 may be divided into sections based on radial strength parameter values and then individually measured using a segmented head or sling apparatus following a procedure such as the procedure described in section 8.1 of the ASTM Standard. The stent 100 strength parameters may also be measured using a hydraulic or pneumatic apparatus following a procedure such as the procedure described in section 8.2 of the ASTM standard. In another example, the stent 100 strength parameters may be measured using finite element analysis.

The strength parameters of the stent 100 may be determined or verified by methods known to those of skill in the art, including through use of a Machine Solutions Inc. RX500 radial force tester equipped with a temperature sensor and environmental air chamber for conducting tests at physiological temperatures.

The stent 100 may be designed to vary the strength parameters of the stent strength profile by: varying the thickness of the elastomeric body 110, varying the thickness of the scaffold 120, using alternative materials for composing each of the elastomeric body 110 and the scaffold 120, or changing the density of the scaffold (i.e., the density of metal mesh), among other things. For example, the radial strength of a body may increase as the thickness increases; stronger materials may increase the radial strength of a body; and increasing the mesh material for a given volume may increase the radial strength of the mesh.

In some embodiments, the stent 100 has an anti-migration surface configured to contact the anatomical lumen 101 and resist the movement of the stent 100 along the anatomical lumen 101. The anti-migration surface may include features to increase the friction between the anatomical lumen 101 and the stent 100. For example, the anti-migration surface may include features such as protrusions or a geometric pattern formed on the outer surface of the elastomeric body 110, or a portion of the scaffold 120 may extend beyond the outer surface of the elastomeric body 110 to anchor the stent 100, among other things.

The anti-migration surface may comprise bumps, protrusions, grooves, or other features. One type of surface pattern may be advantageous for purposes of static friction, while another type of surface pattern may be advantageous for purposes of sliding friction. There may also be properties of the surface pattern that relate to directionality, so as to provide easier or more difficult motion in one direction as compared to another direction. These anti-migration characteristics may vary from place to place on the stent 100.

A portion of the scaffold 120 may partially protrude from the outer surface of the molded body to fixate into the anatomical lumen 101. Another possibility is to have protrusions or a tread-like surface on the outer surface of the elastomeric body. Protrusions serve to reduce the static coefficient of friction of the stent with respect to bodily tissue, and tread serves to reduce the kinetic coefficient of friction of the stent with respect to bodily tissue.

In some embodiments, the outer surface of the stent 100 may have triangle-like protrusions, which may be angled to resist migration in either or both of the axial directions along the lengthwise direction of the stent (either in the proximal direction or in the distal direction).

In some embodiments of the invention, the stent 100 may have a generally circular cross-section at individual places in a section taken perpendicular to a direction that is generally along the longitudinal direction of the stent 100, while at the same time the stent also may be tapered with respect to the lengthwise direction of the stent 100. In some embodiments, the stent may have a shape that is generally of constant cross-section in a section taken perpendicular to a lengthwise direction, but may have a curvature along the lengthwise direction. In some embodiments, the scaffold 120 may have both a taper and a curve.

In some embodiments, the taper and/or curvature may be conducive to being able to insert a molding core into the scaffold lumen that is the interior of scaffold 120, as a preparatory step for molding. Similar considerations apply to removing the core from the stent after completion of molding.

In some embodiments, the taper and/or the curvature may generally correspond to anatomical features of the airway of a patient in whom it is intended that the stent be implanted. In some embodiments, the taper and/or the curvature may generally correspond to anatomical features of healthy individuals, represented with a target anatomical model. In some embodiments, the taper and/or the curvature may correspond to somewhere in between the patient's anatomical features and the anatomical features of a population of individuals with a healthier or more normal anatomical lumen than the patient. In such a situation, the stent can gradually urge the patient's anatomy toward a more desirable configuration. If stents are removed and replaced, it is possible that successive stents can more and more closely approach a desired or normal anatomical configuration.

It should be understood that when the stent 100 is placed into a patient's body, it does not necessarily remain in the patient's body permanently. In many patients and situations, the stent 100 may be removed and replaced at various times. This removal and replacement may be due to reactions of the patient's body to the presence of a foreign object, among other possible reasons. As described elsewhere herein, this removal and replacement also may be used, through a succession of stent designs and dimensions, to gradually encourage a patient's anatomy to shift or re-form toward a desired structural shape that represents normal or non-diseased anatomy.

It should also be understood that placement of the stent 100 may not involve causing a permanent irreversible expansion of the stent 100 after the stent 100 has been placed in its desired location in the patient's body. Sometimes there may be expansion of the stent 100 as it is released from a catheter, but that expansion is usually within the elastic range of deformation, i.e., not permanent or irreversible deformation. It may be possible that the stent 100, for purposes of installation, may exist in a folded or collapsed configuration. However, this may only involve elastic deformation, rather than permanent deformation, in going from the collapsed configuration to the normal configuration or vice versa.

FIG. 5 schematically shows the stent 100 inserted into the anatomical lumen 101 to address an anatomical abnormality 103 narrowing a section of the anatomical lumen 101. In the illustrated embodiment, the anatomical abnormality 103 is a malignant tumor.

To give an example of how the stent 100 would be constructed specific to a patient, suppose a patient has a 10 mm diameter inflamed, curved airway segment requiring a 5 cm long stent. In the middle of the 5 cm segment, there is the malignant tumor 103 expected to reach a size of 1 cm along the airway. While a silicone elastomeric body 110 may be sufficient to address the narrowing of the anatomical lumen 101 due to the inflammation, the segment of the stent 100 aligning with the malignant tumor 103 requires the scaffold 120 to increase the radial strength and offset the force caused by the growth of the tumor. To avoid exerting constant bending pressure on the anatomical lumen 101 by forcing a straight stent into a curved airway, the scaffold 120 and the elastomeric body 110 may have a shape between a target anatomical lumen shape and the patient's anatomical lumen.

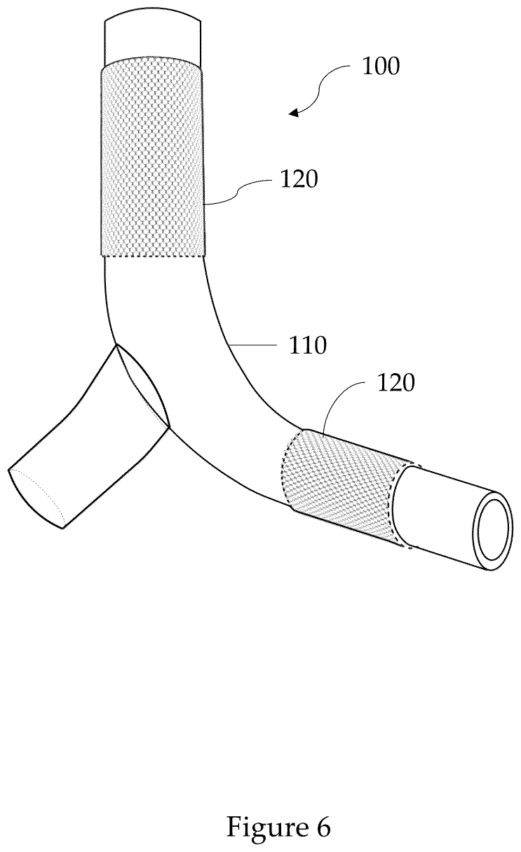

FIG. 6 shows an embodiment of the stent 100 where the elastomeric body 110 includes three opening in a Y-branch configuration. In some embodiments, the scaffold 120 or elastomeric body 110 thickness or other properties may vary from one branch of the “Y” as compared to the other branch of the “Y”. In some embodiments, the thicknesses or other properties of the stent 100 may vary at the point where two or more branches join compared to other portions of the stent 100. If the Y-shaped stent 100 is expected to be deformed in order to fit within an endoscopic instrument, it is possible that specific places of desired thickness could be provided so as to improve the ability of the branches of the “Y” to deform towards each other for the purpose of fitting into the endoscopic instrument. In still other embodiments, there could be provided some other pattern of thickness distribution that contributes to the ability of the stent to be deformed in a desired way for the purpose of fitting into the endoscopic instrument.

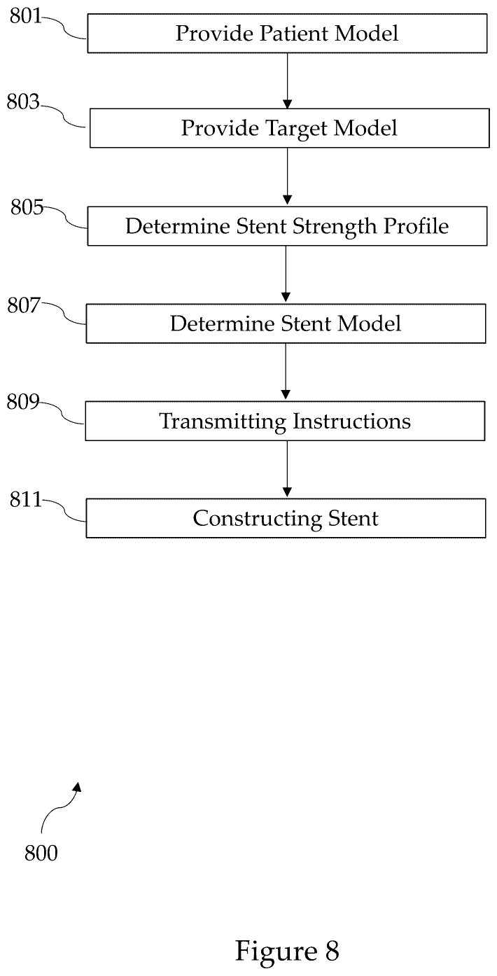

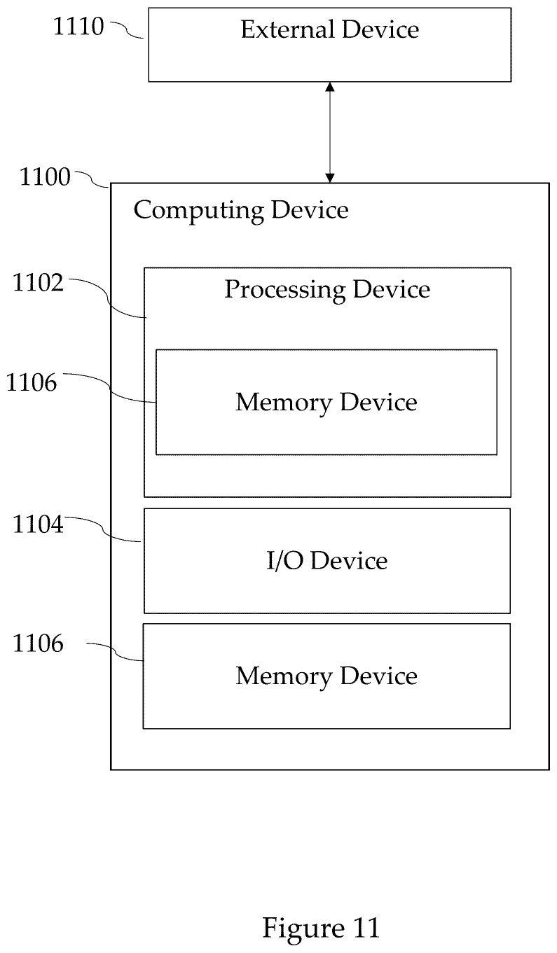

FIG. 8 shows an exemplary process 800 for configuring the stent 100 in accordance with various embodiments. Process 800 may be performed by a computing device in communication with an electronic control system, such as a computing device 1100 illustrated in FIG. 11. It should be further appreciated that a number of variations and modifications to process 800 are contemplated including, for example, the omission of one or more aspects of process 800, the addition of further conditionals and operations, or the reorganization or separation of operations and conditionals into separate processes.

Process 800 begins by providing a patient anatomical lumen model in operation 801. The patient anatomical lumen model may include a 3D model of a patient's anatomy, such as an airway, derived from (segmented) a volumetric medical imaging modality such as a CT scan, MRI, or rotational fluoroscopy, among other things. The images may be uploaded to the computing device and 3D models of the airway may be segmented for use as a template for stent design in order to achieve airway congruency. The patient anatomical lumen model may include representations of anatomical anomalies affecting the anatomical lumen 101.

Process 800 proceeds by providing a target anatomical lumen model in operation 803. Like the patient anatomical model, the target anatomical lumen model may include a 3D model of a patient's anatomy derived from a volumetric medical imaging modality such as a CT scan, MRI, or rotational fluoroscopy, among other things. The target anatomical lumen model may be based on data from one or more healthy patients and aggregated into a single model.

Process 800 proceeds to operation 805, determining a stent strength profile for the stent 100 based on the patient anatomical lumen model and the target anatomical lumen model. The stent strength profile indicates physical characteristics of the stent 100 required to allow stent 100 to treat the patient. In some embodiments, the stent strength profile of the stent 100 allows the stent 100 to manipulate the anatomical lumen 101 to progress towards a shape of the target anatomical lumen model.

For example, determining the stent strength profile may include determining the patient has an anatomical abnormality, such as a tumor, which obstructs or narrows the patient's airway at a certain location. This may include determining the location, type, and/or size of the abnormality. The stent strength profile would then include the strength parameters (i.e., radial stiffness, radial force, or radial strength) required to open the patient's airway by applying radial force on the tumor.

Process 800 then determines a stent model based on the stent strength profile in operation 807. In some treatment strategies, some or all of the cross-sections of the stent 100 may be configured to expand the anatomical lumen 101 by some percentage to correct a narrowing or closure of the anatomical lumen 101. By inserting the stent 100 into position within the anatomical lumen 101, the stent 100 may expand and possibly reconfigure the anatomical lumen 101. In still other treatment strategies, the stent 100 may be chosen to change a curvature of an airway or change a branching angle. Change to the patient's anatomy could relate to the diameter of the anatomical lumen, or a taper angle, or a curvature, or a branching angle.

Determining the stent model may include determining characteristics of the stent 100, scaffold 120, or elastomeric body 110 to meet the required strength parameters of the stent strength profile. These characteristics may include any characteristics of the stent 100, scaffold 120, or elastomeric body 110 mentioned herein, among other things, including: geometric pattern, length, diameter, thickness, material composition, axial curvature, cross-sectional shape, or position of the scaffold 120 relative to the elastomeric body 110.

Operation 807 may include determining the lengths of the elastomeric body 110 and the scaffold 120 or the overlap of the elastomeric body 110 and scaffold 120 in order for the stent 100 to have the required strength to open the airway.

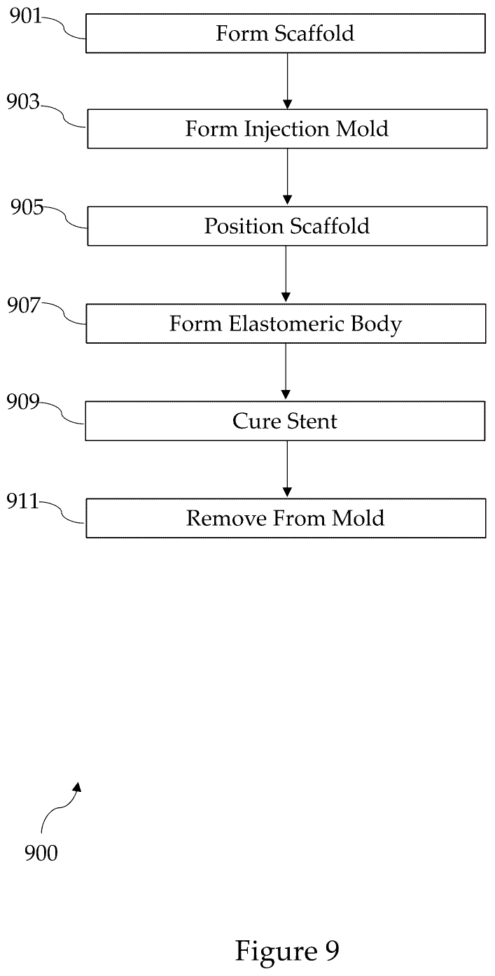

Process 800 then transmits instructions from the computing device to the electronic control system effective to construct the stent 100 in operation 809. The instructions may be transmitted by any means, including a wired connection, a wireless connection, manual user transfer, or transfer through storage device (e.g., USB stick) Finally, process 800 constructs the stent 100 based on the instructions. In some embodiments, the process 800 constructs the stent in the manner described in process 900 of FIG. 9. It should be appreciated that any or all of the foregoing features of process 800 may also be present in the other processes disclosed herein.

FIG. 9 shows an exemplary the process 900 for constructing the stent 100 in accordance with various embodiments. The process 900 may be fully or partially automated by an electronic control system. It should be further appreciated that a number of variations and modifications to the process 900 are contemplated including, for example, the omission of one or more aspects of the process 900, the addition of further conditionals and operations, or the reorganization or separation of operations and conditionals into separate processes.

The process 900 begins at operation 901 where the scaffold 120 is formed. In some embodiments, the structure may be manufactured to collapse while the stent 100 is being moved through a patient into the target anatomical lumen segment.

For the construction of the scaffold, conventional processes that could be used include laser-cutting a metal (possibly extruded) tube. Such processes may, for example, produce a network of struts that are interconnected with each other.

In other embodiments of the invention, the scaffold 120 may be produced by a process such as three-dimensional printing (3DP) onto a bed of metal powder. Such three-dimensional printing may include electron beam welding or laser welding to join groups of powder particles to each other. Such processes may, for example, produce a network of struts that are interconnected with each other.

Three-dimensional printing also may be used to make filaments joined from powder particles in a manner similar to making struts. Using 3DP it is even possible to manufacture filaments that pass over and under each other in a woven configuration. As is usual with powder-based 3DP, after printing, the loose powder particles may be removed by blowing air, by vibration, or by other suitable means. Sintering or a similar post-processing step may also be performed.

The scaffold 120 may be formed by a 3D printer printing the scaffold 120. The construction of the scaffold 120 may be based on the anatomical lumen 101 where the stent 100 will be positioned. For example, the cross-sections of the stent 100 may be based on cross-sections of the anatomical lumen 101 modeled using patient imaging devices. Other characteristics of the scaffold 120 may be based on characteristics of the anatomical lumen 101, such as geometric pattern, length, diameter, material composition, or position of the scaffold 120 relative to the elastomeric body 110, among other things. Characteristics of the anatomical lumen 101 that may affect the construction of the scaffold 120 include patient diagnosis, diameter, axial curvature, shape, or anatomical lumen compressive force or tightness, among other things. In some embodiments, the scaffold 120 axial curvature is designed to follow the axial curvature of the anatomical lumen 101 using a spline algorithm.

The process 900 proceeds to operation 903 where an injection mold is formed with an inner cavity corresponding to the shape of the elastomeric body 110. The inner cavity may also define an anti-migration surface. As with the scaffold 120, the elastomeric body 110 formed by the inner cavity of the injection mold may be based on the anatomical lumen 101 where the stent 100 will be positioned. For example, the cross-sections of the elastomeric body 110 may be based on cross-sections of the anatomical lumen 101. The size and shape of the elastomeric body 110 may be based on characteristics of the anatomical lumen 101, such as length, width, material composition, or a difference between the anatomical lumen 101 and a target anatomical lumen, among other things. Characteristics of the anatomical lumen 101 that may affect the construction of the elastomeric body 110 include patient diagnosis, diameter, shape, or anatomical lumen compressive force or tightness, among other things.

Once a software model of the scaffold 120 has been created, that model may be modified so as to create a model of the stent 100 or elastomeric body 110 having desired features as described herein. Then, a negative of the stent 100 may be created representing the mold cavity. The mold may comprise several mold components. The mold may generally comprise two mold halves that are suitable to be separated from each other at the completion of a molding operation. The two mold halves may typically have a great amount of symmetry with respect to each other and the separation plane of the two mold halves may be arranged to coincide with a plane of symmetry of the article being molded. Because of the presence of a stent lumen as the interior of the stent 100, an appropriate mold core or filler piece may also be created.

In an embodiment of the invention, the mold components may be manufactured by CNC (computer numerical control) machining. Design details such as those described herein can be achieved by manufacturing the mold using CNC machining, in coordination with the design of the scaffold 120,

Alternatively, the mold components, including the mold halves and mold cores associated therewith, may be manufactured by 3D printing. Design details such as those described herein can be achieved by manufacturing the mold using three-dimensional printing, in coordination with the design of the scaffold 120, which may also be manufactured by three-dimensional printing.

The mold may comprise a first outer half and a second outer half. The first and second outer halves may together define all or a substantial part of the outer surface of the stent 100. The two halves may be joinable to each other in a determined relationship to each other for molding, and may be separable from each other for release of the molded part. There may also be an insert or core or a plurality of insert parts or core parts that at least partially define the interior or luminal surface of the molded part. The mold may comprise features for positioning or supporting a core relative to other parts of the mold during the molding process, and for positioning or supporting the scaffold 120 relative to other parts of the mold during the molding process.

The mold components may be designed such that the core is able to be slid into the scaffold 120 prior to molding. Permitted stent geometries to allow this to occur, as discussed elsewhere herein, include cylindrical geometries, tapered or conical geometries, curved geometries, and geometries that are both tapered and curved.

In an embodiment of the invention, the dimensions and other characteristics of the mold may affect or determine the properties of the stent 100 as eventually produced. For example, the dimensions of the mold may affect or determine the thickness of the elastomeric body 110 of the stent 100, i.e., the thickness of the elastomeric body 110.

If the stent 100 is manufactured by a process that includes molding, the design of the mold may also define characteristics of anti-migration surface of the stent 100. These anti-migration characteristics may vary from place to place in the stent 100. These anti-migration features could appear in the elastomeric body 110.

If the stent 100 is manufactured by a process that includes molding and if it is desired that at least some struts or wires or parts of the scaffold 120 partially protrude from the elastomeric body 110, it is possible to make the mold having small recesses corresponding to the eventual position of individual struts or similar features of the scaffold 120. In some embodiments of the invention, the mold can have dimples in it corresponding to where the wires or filaments or struts are expected to partially protrude from the stent 100. Such dimples can help to determine and constrain the position of the scaffold 120 in the mold. It also would be possible to provide inlays that interact with the mold in order to define local details of the outer surface of the elastomeric body 110. The mold can also have patterning to create a migration-resistant surface on the elastomer of the elastomeric body. Although the mold is shown as being divided into halves that are generally symmetric with each other, other mold designs and means of separating the mold are also possible.

The process 900 then proceeds to operation 905, where the scaffold 120 is positioned within the inner cavity of the injection mold. In some embodiments, more than one in scaffold 120 may be positioned within the inner cavity of the injection mold such that the stent 100 will include multiple scaffolds 120 in separate locations that are separated from each other along the longitudinal direction of the stent.

The process 900 then proceeds to operation 907, where viscous elastic material, such as silicone or polyurethane, is injected into the injection mold, forming the elastomeric body 110. The molten material is then allowed to cure in operation 909 until the elastomeric body 110 becomes solidified. The stent 100 may then be removed from the injection mold at operation 911, and the mold core may be removed from the stent 100. It should be appreciated that any or all of the foregoing features of process 900 may also be present in the other processes disclosed herein.

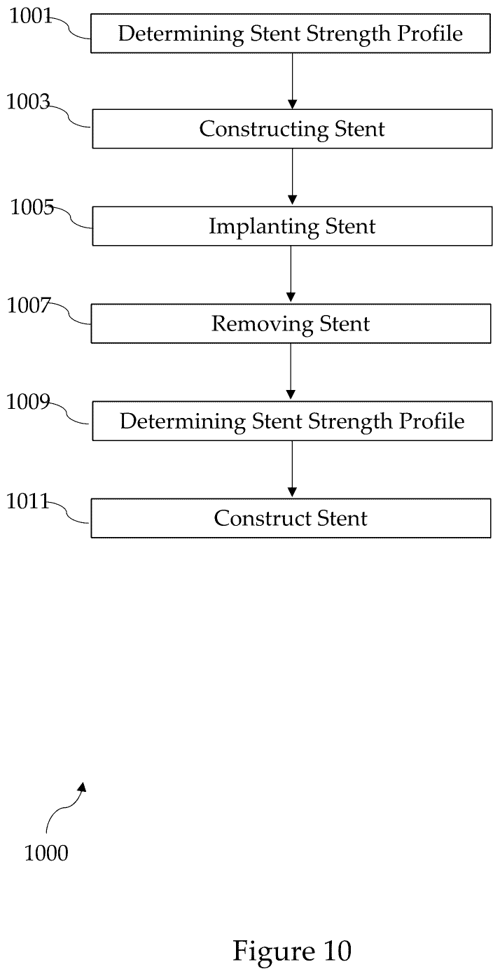

FIG. 10 shows an exemplary the process 1000 for treating the anatomical lumen 101 in accordance with various embodiments. The process 1000 may be fully or partially automated by the computing device 1100 in communication with an electronic control system. It should be further appreciated that a number of variations and modifications to the process 1000 are contemplated including, for example, the omission of one or more aspects of the process 1000, the addition of further conditionals and operations, or the reorganization or separation of operations and conditionals into separate processes.

Process 1000 begins by determining a stent strength profile for the anatomical lumen 101 in operation 1001. The anatomical lumen 101 includes an anatomical abnormality compared to a target anatomical lumen. The anatomical abnormality may be identified by comparing a model of the patient's anatomical lumen to a model of the target anatomical lumen. The stent strength profile has a portion corresponding to the anatomical abnormality which will have an elevated strength to apply a radial force to the anatomical abnormality.

Process 1000 then constructs the stent 100 based on the determined stent strength profile in operation 1003. The stent may be constructed using process 900 described in FIG. 9, among other things.

After constructing the stent, process 1000 proceeds to operation 1005 wherein the stent 100 in implanted in the anatomical lumen 101 of the patient. When implanted, the elevated strength section of the stent 101 is aligned with the anatomical abnormality of the anatomical lumen 101. The stent 100, when implanted, is effective to open the anatomical lumen 101 of the patient.

After a time, anatomical lumen 101 may change as a result of the implanted stent, among other reasons, such that the stent 100 must be replaced. In operation 1007, the stent 100 may be removed from the patient using any known means.

Process 1000 then determines a new stent strength profile based on one or more of the previous stent strength profile, the change to the anatomical lumen, or the target anatomical lumen. The new stent strength profile may allow the anatomical lumen 101 to more closely resemble the target anatomical lumen compared to the changes caused by the first stent. In some embodiments, determining the new stent strength profile may include incrementing a strength parameter so that the shape of the anatomical lumen 101 will more closely resemble the target anatomical lumen compared to the shape caused by the previous stent.

Process 1000 then constructs the stent 100 based on the new stent strength profile in operation 1011. The stent may be constructed using process 900 described in FIG. 9, among other things. It should be appreciated that any or all of the foregoing features of process 1000 may also be present in the other processes disclosed herein.

FIG. 11 schematically shows a computing device 1100 in accordance with various embodiments. The computing device 1100 is one example of an electronic control system which is used to perform one or more operations of the processes disclosed herein, such as processes 700, 800, and 900. The computing device 1100 includes a processing device 1102, an input/output device 1104, and a memory device 1106. The computing device 1100 may be a stand-alone device, an embedded system, or a plurality of devices. Furthermore, the computing device 1100 may communicate with one or more external devices 1110.

The input/output device 1104 enables the computing device 1100 to communicate with an external device 1110. For example, the input/output device 1104 may be a network adapter, a network credential, an interface, or a port (e.g., a USB port, serial port, parallel port, an analog port, a digital port, VGA, DVI, HDMI, FireWire, CAT 5, Ethernet, fiber, or any other type of port or interface), among other things. The input/output device 1104 may be comprised of hardware, software, or firmware. The input/output device 1104 may have more than one of these adapters, credentials, interfaces, or ports, such as a first port for receiving data and a second port for transmitting data, among other things.

The external device 1110 may be any type of device that allows data to be input or output from the computing device 1100. For example, the external device 1110 may be a 3D printer, injection molding machine, a CNC machine, a control system, a sensor, a mobile device, a reader device, equipment, a handheld computer, a diagnostic tool, a controller, a computer, a server, a printer, a display, a visual indicator, a keyboard, a mouse, or a touch screen display, among other things. Furthermore, the external device 1110 may be integrated into the computing device 1100. More than one external device may be in communication with the computing device 1100.

The processing device 1102 may be a programmable type, a dedicated, hardwired state machine, or a combination thereof. The processing device 1102 may further include multiple processors, Arithmetic-Logic Units (ALUs), Central Processing Units (CPUs), Digital Signal Processors (DSPs), or Field-programmable Gate Arrays (FPGA), among other things. For forms of the processing device 1102 with multiple processing units, distributed, pipelined, or parallel processing may be used. The processing device 1102 may be dedicated to performance of just the operations described herein or may be used in one or more additional applications. The processing device 1102 may be of a programmable variety that executes processes and processes data in accordance with programming instructions (such as software or firmware) stored in the memory device 1106. Alternatively or additionally, programming instructions are at least partially defined by hardwired logic or other hardware. The processing device 1102 may be comprised of one or more components of any type suitable to process the signals received from the input/output device 1104 or elsewhere, and provide desired output signals. Such components may include digital circuitry, analog circuitry, or a combination thereof.

The memory device 1106 in different embodiments may be of one or more types, such as a solid-state variety, electromagnetic variety, optical variety, or a combination of these forms, to name but a few examples. Furthermore, the memory device 1106 may be volatile, nonvolatile, transitory, non-transitory or a combination of these types, and some or all of the memory device 1106 may be of a portable variety, such as a disk, tape, memory stick, or cartridge, to name but a few examples. In addition, the memory device 1106 may store data which is manipulated by the processing device 1102, such as data representative of signals received from or sent to the input/output device 1104 in addition to or in lieu of storing programming instructions, among other things. As shown in FIG. 11, the memory device 1106 may be included with the processing device 1102 or coupled to the processing device 1102, but need not be included with both.

It is contemplated that the various aspects, features, processes, and operations from the various embodiments may be used in any of the other embodiments unless expressly stated to the contrary. Certain operations illustrated may be implemented by a computer executing a computer program product on a non-transient, computer-readable storage medium, where the computer program product includes instructions causing the computer to execute one or more of the operations, or to issue commands to other devices to execute one or more operations.

While the present disclosure has been illustrated and described in detail in the drawings and foregoing description, the same is to be considered as illustrative and not restrictive in character, it being understood that only certain exemplary embodiments have been shown and described, and that all changes and modifications that come within the spirit of the present disclosure are desired to be protected. It should be understood that while the use of words such as “preferable,” “preferably,” “preferred” or “more preferred” utilized in the description above indicate that the feature so described may be more desirable, it nonetheless may not be necessary, and embodiments lacking the same may be contemplated as within the scope of the present disclosure, the scope being defined by the claims that follow. In reading the claims, it is intended that when words such as “a,” “an,” “at least one,” or “at least one portion” are used there is no intention to limit the claim to only one item unless specifically stated to the contrary in the claim. The term “of” may connote an association with, or a connection to, another item, as well as a belonging to, or a connection with, the other item as informed by the context in which it is used. The terms “coupled to,” “coupled with” and the like include indirect connection and coupling, and further include but do not require a direct coupling or connection unless expressly indicated to the contrary. When the language “at least a portion” or “a portion” is used, the item can include a portion or the entire item unless specifically stated to the contrary. Unless stated explicitly to the contrary, the terms “or” and “and/or” in a list of two or more list items may connote an individual list item, or a combination of list items. Unless stated explicitly to the contrary, the transitional term “having” is open-ended terminology, bearing the same meaning as the transitional term “comprising.”

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented as a pre-configured, stand-along hardware element and/or as preprogrammed hardware elements (e.g., application specific integrated circuits, FPGAs, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

The embodiments of the invention described above are intended to be merely exemplary; numerous variations and modifications will be apparent to those skilled in the art. Such variations and modifications are intended to be within the scope of the present invention as defined by any of the appended claims. It shall nevertheless be understood that no limitation of the scope of the present disclosure is hereby created, and that the present disclosure includes and protects such alterations, modifications, and further applications of the exemplary embodiments as would occur to one skilled in the art with the benefit of the present disclosure.

What is claimed is:

1. A method for constructing a stent, comprising:
providing a patient anatomical lumen model;
providing a target anatomical lumen model;
determining a stent strength profile for the stent based on the patient anatomical lumen model and the target anatomical lumen model;
determining a stent model including an elastomeric body model and a scaffold model based on the stent strength profile; and
transmitting instructions to an electronic control system effective to construct the stent using the stent model, the stent including an elastomeric body corresponding to the elastomeric body model and a scaffold corresponding to the scaffold model, the scaffold comprising a metallic material.

2. The method of claim 1, wherein the stent model includes a plurality of cross-sections, and wherein the stent strength profile includes a strength parameter having varying values corresponding to each of the plurality of cross-sections.

3. The method of claim 2, wherein the strength parameter is a radial stiffness parameter or a radial force parameter.

4. The method of claim 1, wherein determining the stent model includes determining an elastomeric body model length based on the stent strength profile and determining a scaffold length based on the stent strength profile.

5. The method of claim 1, wherein determining the elastomeric body model and the scaffold model based on the stent strength profile includes determining a position of the elastomeric body model relative to the scaffold model.

6. The method of claim 1, comprising: constructing the stent in response to transmitting the instructions.

7. The method of claim 1, wherein determining the stent strength profile includes determining an anatomical abnormality position, and wherein determining the elastomeric body model and the scaffold model based on the stent strength profile includes determining a scaffold model position relative to the elastomeric body model based on the anatomical abnormality position.

8. A patient-specific stent, comprising:
an elastomeric body; and
a scaffold coupled to the elastomeric body and including a metallic material,
wherein the patient-specific stent is configured to include a stent strength profile along a length of the patient-specific stent, the stent strength profile including an elevated strength section.

9. The patient-specific stent of claim 8, wherein the patient-specific stent includes a first cross-section formed of the elastomeric body and a second cross-section parallel to the first cross-section, and wherein a radial stiffness of the second cross-section is greater than a radial stiffness of the first cross-section.

10. The patient-specific stent of claim 8, wherein the stent strength profile is a function of an elastomeric body length relative to a scaffold length.

11. The patient-specific stent of claim 8, wherein a location of the elevated strength section of the stent strength profile is a function of a scaffold position relative to the elastomeric body.

12. The patient-specific stent of claim 8, wherein the elastomeric body comprises a least one of silicone or polyurethane.

13. The patient-specific stent of claim 8, wherein the stent strength profile includes a first radial stiffness parameter corresponding to an anatomical abnormality cross-section of the stent, and a second radial stiffness parameter corresponding to another cross-section of the stent, wherein the first radial stiffness parameter is at least two times greater than the second radial stiffness parameter.

14. The patient-specific stent of claim 8, wherein the stent strength profile includes a varying radial stiffness as a function of at least one of: a varying thickness of the elastomeric body, a difference between an elastomeric body length and a scaffold length, a varying composition of the elastomeric body or the scaffold, a varying geometric pattern of the scaffold, or a position of the scaffold relative to the elastomeric body.

15. A method for treating an anatomical lumen, comprising:
determining a first stent strength profile based on a patient anatomical lumen and a target anatomical lumen, the first stent strength profile including a first elevated strength section;
constructing a first stent based on the first stent strength profile, the first stent including an elastomeric body coupled to a metallic scaffold;
implanting the first stent in the patient anatomical lumen, wherein the elevated strength section of the stent strength profile is configured to align with an anatomical anomaly;
removing the stent;
determining a second stent strength profile based on the first stent strength profile and the patient anatomical lumen, the second stent strength profile including a second elevated strength section; and
constructing a second stent based on the second stent strength profile.

16. The method of claim 16, wherein the stent model includes a plurality of cross-sections, and wherein the stent strength profile includes a strength parameter having varying values corresponding to each of the plurality of cross-sections.

17. The method of claim 16, wherein the strength parameter is a radial stiffness parameter or a radial force parameter.

18. The method of claim 15, wherein the patient-specific stent includes a first cross-section formed of the elastomeric body and a second cross-section parallel to the first cross-section, and wherein a radial stiffness of the second cross-section is greater than a radial stiffness of the first cross-section.

19. The method of claim 15, wherein the stent strength profile is a function of an elastomeric body length relative to a scaffold length.

20. The method of claim 15, wherein a location of the elevated strength section of the stent strength profile is a function of a scaffold position relative to the elastomeric body.
